(19) United States
(12) Patent Application Publication
(10) Pub. No.: US 2016/0367480 A1
(43) Pub. Date: Dec. 22, 2016

(54) VITAMIN C DELIVERY SYSTEM AND LIPOSOMAL COMPOSITION THEREOF

(71) Applicant: Joseph Jongeul JEUNG, Diamond Bar, CA (US)

(72) Inventor: Joseph Jongeul JEUNG, Diamond Bar, CA (US)

(21) Appl. No.: 15/185,761

(22) Filed: Jun. 17, 2016

(30) Foreign Application Priority Data
Jun. 18, 2015 (KR) 10-2015-0086290

(51) Int. Cl. A61K 9/127; A61K 31/375

(52) U.S. Cl. CPC A61K 9/127; A61K 31/375

(57) ABSTRACT

The present invention relates to a vitamin C delivery system and liposomal composition thereof. The liposome composition of vitamin C of the present invention consists of lecithin of the sunflower and therefore, improves stability and bioavailability of vitamin C. In addition, the composition does not use soybean lecithin thereby resolving side effect thereof.

### TECHNICAL FIELD

The present invention relates to a vitamin C delivery system and liposomal composition thereof and more particularly relates to a liposome composition of vitamin C consisted of lecithin of the sunflower and the delivery system thereof.

### BACKGROUND ART

Vitamin C (ascorbic acid) improves immunity within the human body and when it is applied to the skin, it promotes formation of collagen which is a component of cartilage, capillary and muscle and therefore prevents skin damage caused by UV light and the production of wrinkles. It also prevents histamine production which induces allergic reactions and inhibits formation of melanin which causes skin aging. However, vitamin C is especially sensitive to the external environments such as oxygen, heat and light and therefore more prone to oxidation decomposing. These oxidation include two electron transfer processes and electrolysis of the hydrogen ion. As a result, Dehydro ascorbate radicals are formed as an oxidation intermediate and cause decomposition. Accordingly, it is not stable and used as a small dose of active ingredients in medicine, food and cosmetic field.

This vitamin C is taken as forms of tablet, capsule, powder and liquid, but it is not absorbed to the body effectively.

To improve the stability of vitamin C, U.S. Pat. No. 4,938,969 and EUP 533,667 B1 disclosed methods of adding antioxidants, two methods which are stabilizing vitamin C with multi-lamellar emulsion, and stabilizing vitamin C using oil-in-water emulsion in terms of formulation. In addition, to improve the stability of vitamin C itself, a method of modifying vitamin C structure with SA (Sodium ascorbylphosphate), MAP (Magnesium ascorbylphosphate), CAP (Calcium ascorbylphosphate), AAP (ascorbic acid polypeptide), EAE (Ethyl ascorbic ether) and AD (Ascorbyl Dipalmate), AEP (Ascoly ethylsilanol pectinate) have been suggested.

In addition to the above-mentioned methods, to improve the stability of vitamin C itself, methods to prevent Vitamin C decomposition caused by the external environments such as water, oxygen, heat and light using physiochemical binding of the vitamin C to high molecular chains and encapsulating into nano-type micro foam, namely the liposome, has also been recently suggested.

As an example of the materials for stabilizing vitamin C, cationic polymers and anionic polymers are well known. For example, anionic polymers are preferred which can enfold vitamin C in three dimensions isolating it from water and air and enabling it to absorb UV light. The anionic polymers may include monosaccharides, polysaccharides, cellulose, gelatin, hyaluronic acid, alginic acid, starch, and carboxymethyl cellulose (CMC). Cationic polymers should be able to be formed into vitamin C with an acid-base binder. For example, it may be a polymethylmethacrylate copolymer or a styrene copolymer comprising chitosan, polyethyleneimine, polyvinylpyrrolidone, amino acids or 4 kinds of ammonium (KR 10-0588464).

A liposome consists of vesicular structure based on lipid bilayer enfolding liquid composition comprised of phosphatide or sterol. Therefore, liposomes show variable physicochemical properties according to their sizes and surface charge. Recently, attention was drawn to liposomes as pharmaceutically acceptable carriers for diagnosis or treatment for diseases. Especially, research on a liposomes used as drug delivery systems have been dramatically developed, and in this regard, a Korean Publication No. 10-2011-71017 discloses a pharmaceutical composition and a formula for gene therapies of various diseases in contact with an active solution with a solution comprising amino acid compound or lipid in organic solvent to improve the efficacy of the delivery of carrier and producing collision stream. However, a liposome may induce agglutination of blood by interacting with various plasma proteins and can be captured by reticuloendothelial systems (RES). For example, the Kupfer cell in the liver or the fixed macrophage in the spleen may capture liposomes before they reach the target and this capture by RES inhibits selective transportation of liposome to target tissues or cells. In addition, liposomes are unstable as they are easy to make electrostatic reactions, hydrophobic reactions and van der waals reactions with plasma protein which may lead to a rapid removal from the ER before reaching the target during blood circulation. Moreover, further to the interaction of liposome with cells or proteins, the drug itself can interact with a lipid of the liposome which makes the capsulation difficult. Therefore, a liposome is unstable during its manufacturing process and the drug may leak before arriving at a tumor site and can be decomposed by cytotoxicity. Consequently, the researchers tried to explore long time-circulating liposomes which are able to be released within blood vessel only. The effort to transport a drug to a target region by extending circulation time of liposomes is disclosed in U.S. Pat. No. 4,501, 725. Furthermore, U.S. Pat. No. 4,920,016 discloses a method of hardening liposomal membrane using neutral phosphatides, and U.S. Pat. No. 6,083,530 discloses a method of formulating liposomes which enables the increase of the ratio of lipid to drugs up to 3-80 folds, and U.S. Pat. No. 6,132,763 discloses a method of inducing phosphatides with polyethylene glycol (PEG) and pegylated liposome.

In U.S. Pat. No. 6,132, 763, a surface of a liposome is coated with hydrophilic polymer such as polyethylene glycol (PEG) and therefore it prevent adhesions of various plasma proteins. The inventors named it stealth liposome. However, a formula comprising the pegylated phosphatides shows toxicity of the hand-foot syndrome causing a skin rash or ulcer (Kenneth B. Gordon, Cancer, Vol 75(8), 1995, 2169-2193).

Meanwhile, liposome compositions with high bioaffinity may cause problems such as bring changes of color and scent which results from extraction of natural materials.

The cell penetration ratio, of a liposome formula using phospholipids as a surfactant is formed to be increased (Biochem, Biophys. Acta, 1237, (1995)), but it has disadvantage of having an unstable structure since it consists of phospholipids only. Moreover, liposome formulas prepared using soybean lecithin or egg lecithin have high cell penetration ratio by penetrating into gaps between skin keratinocytes, however, the hardness of the structure is weak and therefor causes oxidation of double binding regions of unsaturated lecithin by oxygen, metallic ions and more. Therefore, the structure of the liposome is destroyed which causes changes in color and scent (Biophsics J. vol. 79 No. 1(2000): 328-339).

According to the inventor's research, the liposome formula made of sunflower lecithin has appropriate hardness for a stable liposome structure to prevent changes in color and scent, and also appropriate rigidity to enhance cell penetration ratio and affinity. Moreover, compared to soybean lecithin, sunflower lecithin does not have a risk of having characteristics of GMO (Genetically Modified Organism) and side effects such as thyroid related diseases, interruption of mineral uptake or soybean caused allergies. Especially, because as it is extracted by cold pressing and not by extraction with organic solvents, it does not have side effects caused by solvent extraction.

So far, there is no report regarding a vitamin C liposome composition using the liposome consisting of sunflower lecithin so as to uptake vitamin C stably into body.

### TECHNICAL PROBLEM

Accordingly, the object of the present invention is to provide a vitamin C liposome composition using the liposome consisting of sunflower lecithin. Another object of the present invention is to provide vitamin C delivery system improving bioavailability by administrating the liposome formula orally to be absorbed into blood circulation system.

### TECHNICAL SOLUTION

The object of the present invention is achieved by providing a vitamin C liposome composition comprising 500 mg of vitamin C, 100 mg of sunflower lecithin, 2 mg of candelilla wax, 288 mg of medium chain triglyceride (MCT), 100 mg of Glycerin and 10 mg of distilled water prepared by the following steps:
preparing a water phase compound forming liposomes by mixing vitamin C, glycerin and sunflower lecithin with distilled water followed by stirring;
the 1st emulsifying step that medium chain triglyceride (MCT) and candelilla wax is added to the water phase compound and stirred for 1 hr;
preparing a vitamin liposome composition through the 2^{nd} emulsifying step wherein the emulsion is homogenized with a homogenizer at 5000 rpm for 2 hrs;
preparing soft or hard capsules as a product by filling them with the vitamin C liposome composition; and
determining the stability and bioavailability of the composition by measuring titer and bioavailability of the vitamin C.

### ADVANTAGEOUS EFFECT

The present invention is advantageous in that it provides a liposome composition improving stability and bioavailability of vitamin C. In addition, the composition does not use soybean lecithin thereby resolving side effect thereof.

### DESCRIPTION OF DRAWINGS

FIG. 1 demonstrates the vitamin C liposome structure manufactured according to the Example of the present invention.

FIG. 2a-2d represents the test results showing bioavailability of vitamin C after uptake of the vitamin C liposome composition of the present invention

FIG. 3 is a graph which shows results of comparative experiments measuring bioavailability of the vitamin C after uptake of the vitamin C liposome of the present invention with a vitamin C liposome consisting of conventional vitamin C powder and soybean lecithin.

X: Dr. JEUNG, JONG; administered 0.5 g of the inventive products (66.43 μM/L)

V: Dr. SHAH, HITENDRA; administered 1 g of the inventive products (103.9 μM/L)

602 : BARRERA, JUANITA: administered 2 g of the inventive products (135.9 μM/L)

□: Dr. JEUNG, JONG; administered 3 g of the inventive products (181.5 μM/L)

### MODE FOR INVENTION

Hereafter, the present invention would be described in detail through the examples and experimental examples. However, the description certainly does not limit the scope of this invention.

The vitamin C liposome composition of the present invention is produced by the steps comprising as follows:
(a) preparing a water phase compound forming liposomes by mixing vitamin C, glycerin and sunflower lecithin with distilled water followed by stirring;
(b) the 1st emulsifying step that medium chain triglyceride (MCT) and candelilla wax is added to the water phase compound and stirred for 1 hr;
(c) preparing a vitamin liposome composition through the 2^{nd} emulsifying step where the emulsion is homogenized with a homogenizer at 5000 rpm for 2 hrs;

The significant point of the present invention is to prepare vitamin C filled liposomes using sunflower lecithin. Although methods of preparing liposomes are well known in the art, huge differences may occur in stability and biological characters according to liposome formable materials, solvents and the materials to be filled within liposomes, and its order when adding, the ratio of its composition and conditions of stirring.

According to the present invention, the oil ingredient from the oil phase of the step (b) can be selected from the group consisting of paraffin oil, α-bisabolol, stearyl glycyrrhetinate, salicylic acid, tocopheryl acetate, panthenol, glyceryl stearate, cetyl octanolate, isopropyl myristate, 2-ethylene isopelagonate, di-c12-13 alkyl malate, ceteatyl octanoate, butylene glycol dicaptylate, butylene glycol dicaprate, isononyl isostearate, isostearyl isostearate, triglycerides, beeswax, canauba wax, suctose distearate, PEG-8 beeswax, candelilla (euphorbia cerifera) wax, mineral oil, squalene, squalane, monoglyceride, diglyceride, triglyceride, middle chain glyceride, myglyol and cremophor. Also, medium chain triglyceride (MCT) is preferred for using as a subadditive and candelilla wax is preferred for using as an emulsifier.

In addition, the vitamin C liposome compositions may be formed for oral administrations such as a tablet, a capsule and powder, but it is preferably formed, it may be formed into a soft or hard capsules with filling. The formula of the present invention may comprise conventional additives and excipients within the capsule formula. For example, it may be a humectant, a pH control agent, a metal chelating agent, a viscosity increasing agent and distilled water, but it is not limited thereto.

Hereafter, the present invention will be described in detail by examples. However, the description does not limit the scope of the invention.

### EXAMPLE 1

### Preparation of a Vitamin C Liposome Composition

The vitamin C liposome compositions of the present invention preferably have the component ratio described in Table 1.

TABLE 1
Component ratio of the vitamin C liposome compositions

| Criteria | | | Compound | Amount |
|---|---|---|---|---|
| Vitamin C | Water-phase | Vitamin C | Ascorbic acid | 500 mg |
| liposome | Compound | Phosphatide | Sunflower | 100 mg |
| compositions | (liposome) | Preservative | lecithin | |
| | | | Glycerin | 100 mg |
| | | Distilled water | Pure water | 10 mg |

| | | |
|---|---|---|
| Emulsifier | Candelilla wax | 2 mg |
| Sub-additive | Medium chain | 288 mg |
| | triglyceride | |
| | (MCT) | |

| | |
|---|---|
| Weight in total | 1000 mg |

The inventors of the present application prepared a vitamin C liposome composition according to the component ratio of Table 1. First, they added 100 mg of glycerin, 500 mg of vitamin C and 100 mg of sunflower lecithin in a flask with 10 mg of distilled water and stirred for 30 mins at 30 ° C. and therefore prepared a water phase compound filled with vitamin C in a liposome. In the above-mentioned flask, they added 288 mg of MCT as a sub-additive and 2 mg of candelilla wax as an emulsifier and stirred for 1 hr for the 1^{st} emulsification. The product of the 1^{st} emulsification were emulsified by homogenization for 2 hrs at 5,000 rpm so as to be appeared milky for the 2nd emulsification thereby preparing the vitamin C liposome composition of the present invention. The above-mentioned vitamin C liposome composition was prepared into a vitamin C liposome formula by filling it into a soft capsule. This formula was used for the material in the examples described below.

### COMPARATIVE EXAMPLE 1-3

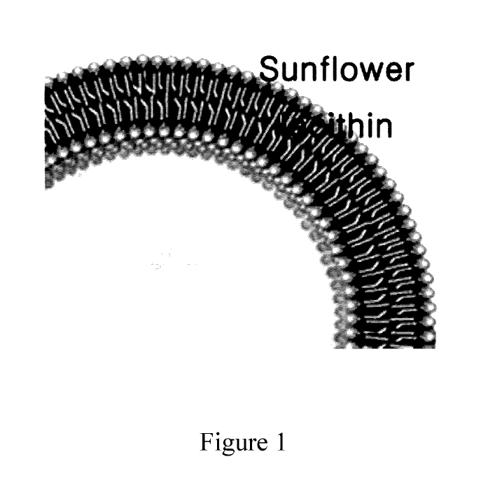

The inventors prepared capsules according to the example 1 with paraffin oil instead of candelilla wax (comparative example 1), ethanol instead of glycerin (comparative example 2) and soybean lecithin instead of sunflower lecithin (comparative example 3). The scheme of sunflower lecithin liposome structure of the vitamin C liposome structure according to the present invention is shown in FIG. 1.

### EXPERIMENTAL EXAMPLE 1

### Titration of Vitamin C

According to the example 1 and the comparative Example 1-3, vitamin C titer of the capsulated products were measured and compared. The results are shown in Table 2. The titers of vitamin C of each products were measured at 36.5 ° C. (body temperature) and 45° C. (Shelf temperature) after one month later from the production of each product. The results are calculated using the [Equation 1] below. The titers were measured by quantification of remaining vitamin C using HPLC (Waters Co. LTD.) To determine the amount of remaining vitamin C, the used wave length of detector was 254 nm, the column was Luna C18 column of phenomenex Co., Ltd. and the flow rate was 0.8 mL/min. The comparative amount of vitamin C was determined by drawing a standard measurement graph using the measured remaining amount and the peak of UV spectrophotometer at 266 nm. The spectrophotometer used was Helios β, a product of spectronic unicam Co. Ltd.

TABLE 2
Vitamin C Titers

| | | Comparative | | |
|---|---|---|---|---|
| | | Experiments | | |
| Condition | Example 1 | 1 | 2 | 3 |
| Room Temperature | 95 | 93 | 93 | 95 |
| 36.5° C. | 94 | 91 | 90 | 93 |
| 45° C. | 93 | 87 | 85 | |

A=titers of vitamin C of each product after 1 month post-production/primary titers of vitamin C of each product×100 (1)

As seen in Table 2, when the titers of the product of the Example 1 and the products of the comparative example 1-3 are compared, the inventive product shows improved effect on vitamin C stability at the temperature of 36.5 ° C. (body temperature) and 45° C. (Shelf temperature).

### EXPERIMENTAL EXAMPLE 2

### Bioavailability of the Inventive Vitamin C Liposome Product

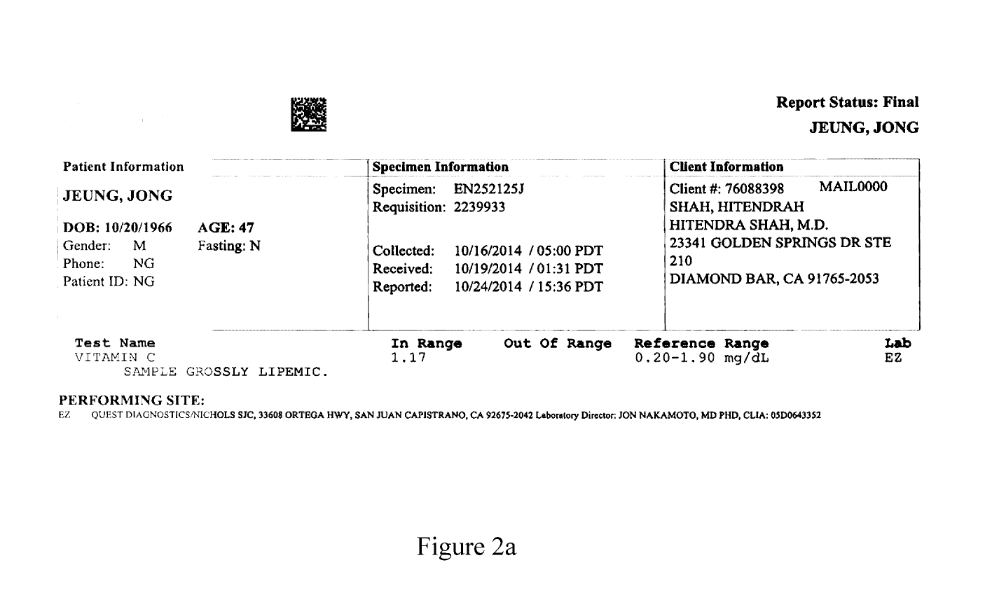

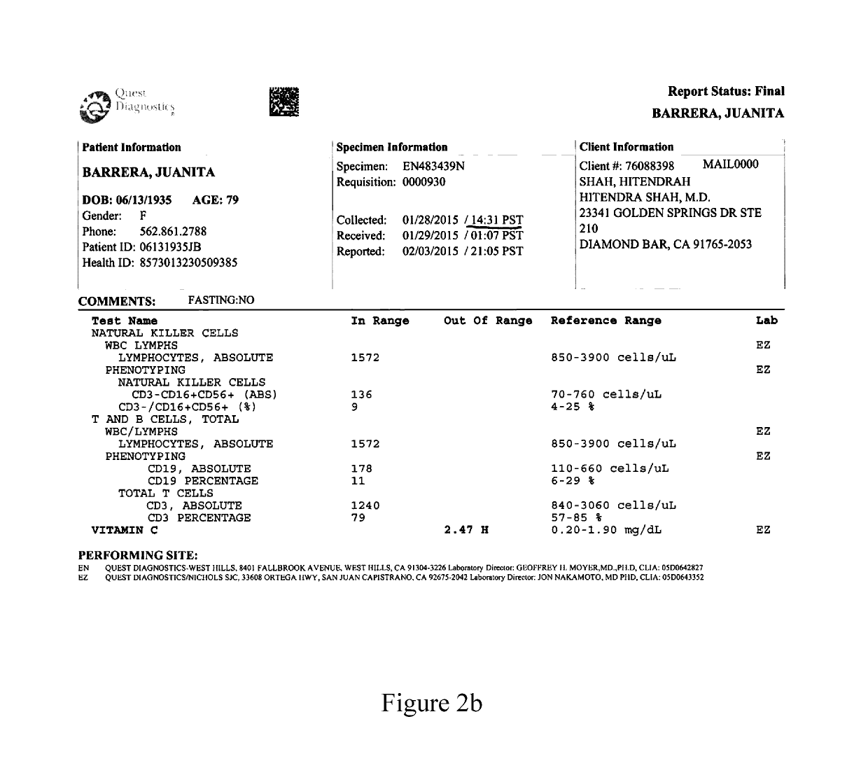

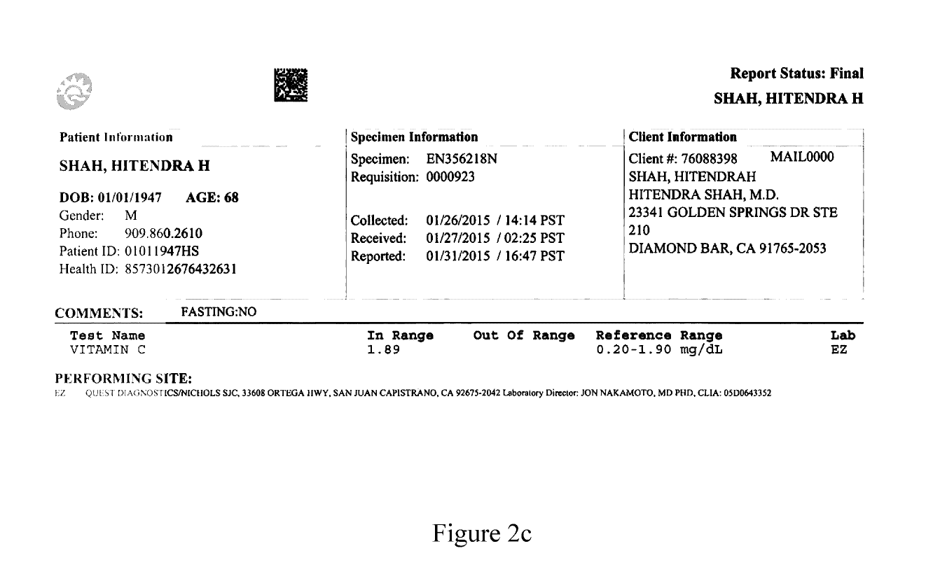

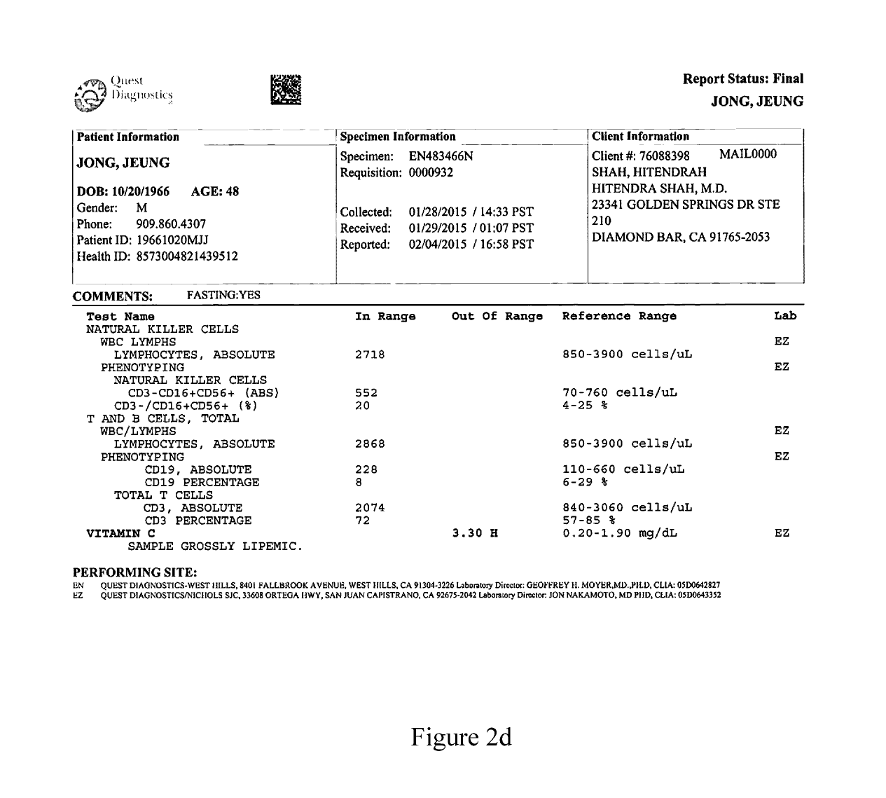

The inventors performed clinical trials using with a vitamin C liposome product prepared through Example 1. The persons participating in the trial were Dr. JEONG, JONG (Male, age 47), BARRERA, JUANITA (Female, age 79) and Dr. SHAH, HITENDRA H (Male, age 68). In addition, Dr. JEONG, JUNG was administered with 3 g of the product orally on Jan. 28, 2016, whereas the dose used in the clinical trial was 0.5 g. The blood test was performed after 4 hrs and 15 mins post administration to confirm the stability of the product. The above clinical trials were requested to Quest Diagnostics and the results are shown in FIG. 2a-2d. In case of the Dr. JEONG, the measurement of vitamin C after 4 hrs and 15 mins of post- administration of 0.5 g of the product was 1.17 mg/dL (FIG. 2a) and therefore, 66.43 uM/L (1.17 mg/dL×55) of vitamin C was remaining. Likewise, in case of Ms. BARRERA, the measurement of vitamin C after 4 hrs and 15 mins of post-administration of 1 g of the product was 2.47 mg/dL (FIG. 2b) and therefore, 135.9 uM/L (2.47 mg/dL×55) of vitamin C was remaining. In the case of Dr. SHAH, the measurement of vitamin C after 4 hrs and 15 mins of post-administration of 1 g of the product was 1.89 mg/dL (FIG. 2c) and therefore, 103.9 uM/L (1.89 mg/dL×55) of vitamin C was remaining. Meanwhile, in case of Dr. JEONG, the measurement of vitamin C after 4 hrs and 15 mins of post-administration of 3 g of the product was 3.3 mg/dL (FIG. 2d) and therefore, 181.5 uM/L (3.3 mg/dL×55) of vitamin C was remaining. Lastly, there were no side effects such as vomiting, fever and dizziness when the testees were administered with the inventive product.

### EXPERIMENTAL EXAMPLE 3

### Comparison on the Bioavailability of the Vitamin C Liposome Product of the Present Invention with the Conventional Vitamin C Powder

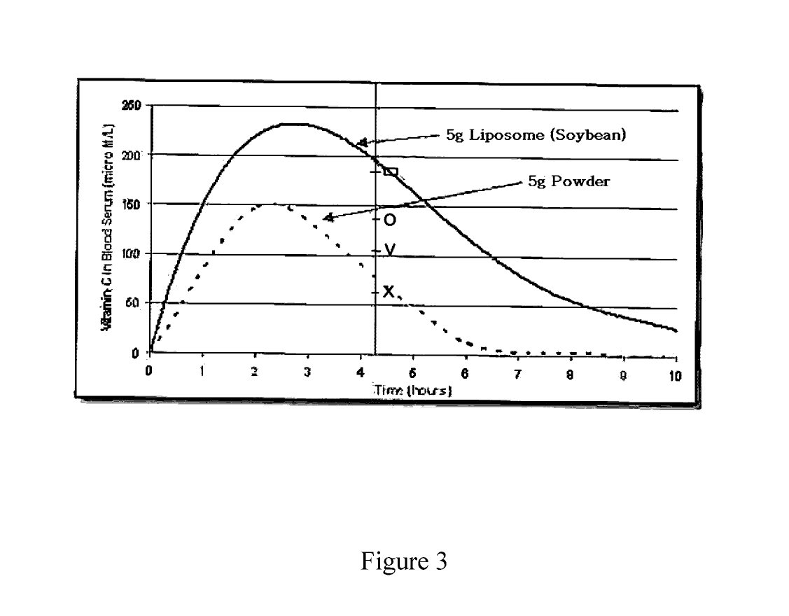

The clinical data on bioavailability of the inventive vitamin C liposome product (Experimental Example 2) were compared with the conventional vitamin C liposome product which consists of vitamin C-powder and soybean lecithin (Empirical Labs Liposomal Vitamin C). As shown in FIG. 3, in case of Dr. JEONG, the bioavailability of vitamin C measured after administration of 0.5 g of the inventive product (x) was equivalent as that of 5 g of the conventional vitamin C powder. In the case of Dr. Shah, the bioavailability of vitamin C measured after administration of 1 g of the inventive product (v) was even higher than that of 5 g of the conventional vitamin C powder. In addition, in the case of Dr. JEONG, the bioavailability of vitamin C measured after administration of 3 g of the inventive product (□) was similar to that of 5 g of the product consisting of soybean lecithin. In conclusion, the inventors confirmed that the sunflower lecithin used to formulate vitamin C liposome product aids in showing improved bioavailability than using soybean lecithin.

### INDUSTRIAL APPLICABILITY

The present invention uses sunflower lecithin as a component of the vitamin C liposome composition increasing the bioavailability, improvement of stability, and the titer measurement of vitamin C. In conclusion, the present invention is useful for the functional health food industry.

1. A method for preparing a vitamin C liposome composition produced by the steps comprising as follows:
(a) preparing a water phase compound forming liposomes by mixing vitamin C, glycerin and sunflower lecithin with distilled water followed by stirring;
(b) the 1^{st} emulsifying step that medium chain triglyceride (MCT) and candelilla wax is added to the water phase compound and stirred for 1 hr;
(c) preparing a vitamin liposome composition through the 2^{nd} emulsifying step where the emulsion is homogenized with a homogenizer at 5000 rpm for 2 hrs;

2. A vitamin C liposome composition according to the method of claim 1.

3. The vitamin C liposome composition of claim 2, wherein 1000 mg of the total composition consists of 500 mg of vitamin C, 288 mg of medium chain triglyceride (MCT), 2 mg of candelilla wax, 100 mg of Glycerin, 100 mg of sunflower lecithin, and 10 mg of distilled water.

4. The vitamin C liposome composition of claim 2, wherein the formula used for the composition is soft capsule or hard capsule.

5. A vitamin C delivery system which is characterized by oral administration of the vitamin C liposome composition of claim 4.
